(19) United States
(12) Patent Application Publication
(10) Pub. No.: US 2025/0122135 A1
(43) Pub. Date: Apr. 17, 2025

(54) PURIFICATION OF AN ALCOHOL DEHYDRATION PRODUCT STREAM

(71) Applicant: UOP LLC, Des Plaines, IL (US)

(72) Inventors: Ankita Gupta, Gurugram (IN); Akarsha Srivastava, Gurugram (IN); Silpa S, Gurugram (IN); Nilesh Tekawade, Gurugram (IN); Priyesh Jayendrakumar Jani, Gurugram (IN); Gautam Pandey, Gurugram (IN); Thomas Calabrese, Hoffman Estates, IL (US); Amanda Hickman, Evanston, IL (US); Deng-Yang Jan, Elk Grove Village, IL (US); Jayant Gorawara, Buffalo Grove, IL (US); Nickolas Kapaun, Chicago, IL (US); Jeannie Mee Blommel, Oregon, WI (US); Rodrigo Lobo, Des Plaines, IL (US)

(21) Appl. No.: 18/910,879

(22) Filed: Oct. 9, 2024

Related U.S. Application Data
(60) Provisional application No. 63/590,041, filed on Oct. 13, 2023.

(51) Int. Cl. C07C 7/12; B01D 53/62; B01D 53/82; B01D 53/86; B01J 20/06; B01J 23/72; C01B 5/00; C01B 32/50

(52) U.S. Cl. CPC C07C 7/12; B01D 53/62; B01D 53/82; B01D 53/8671; B01J 20/06; B01J 23/72; C01B 5/00; C01B 32/50; B01D 2256/24; B01D 2257/108; B01D 2257/502

(57) ABSTRACT

A process is provided for treating a stream comprising light olefins and carbon monoxide by passing the stream through a bed containing a copper containing material. The carbon monoxide needs to be removed in order to prevent poisoning of catalysts in downstream oligomerization reactors. This carbon containing material converts the carbon monoxide and hydrogen to carbon dioxide and water. The olefin stream is then dried and sent to an oligomerization step. There may be three beds so that the beds may alternate regeneration, reaction with CO and H₂ and scavenging O₂ steps.

This relates to removal of carbon monoxide from a product stream from an alcohol dehydration reactor. More particularly, this relates to the addition of oxygen to regenerate a copper containing material while preventing carbon monoxide, oxygen and hydrogen from going downstream.

### BACKGROUND

Light olefins are produced from alcohol dehydration before the light olefins are oligomerized in a step leading to sustainable aviation fuel. The main steps after the dehydration include the oligomerization of the light olefins to olefins that are in the range of about C₈ to C₁₆ and then hydrogenation of the olefins to make paraffins. The gas stream from the dehydration unit may be compressed via 4-stage compressors before being sent to the oligomerization section. In the case of the dehydration of ethanol, the ethanol dehydration effluent gas stream is composed of about 98 wt. % ethylene and the remaining approximately 2 wt. % consists of carbon monoxide, carbon dioxide, hydrogen, other olefins and oxygenates. While constituents such as carbon dioxide and hydrogen, do not inhibit the oligomerization process, carbon monoxide deactivates the oligomerization catalyst.

In the past, carbon monoxide has been removed from an ethylene stream by fractional distillation. However, under the conditions encountered herein, the much higher levels of CO and the presence of hydrogen would result in a very short catalyst life without use of the process herein.

### SUMMARY

The dehydration of alcohols is an important step in the process of converting alcohols to jet fuels. In particular, an ethanol dehydration reaction is a key step in the process of converting ethanol to jet fuel. Ethanol is dehydrated to ethylene but carbon monoxide is one of the byproducts of the process. Carbon monoxide is harmful to the performance of downstream oligomerization catalysts which results in the requirement that the carbon monoxide must first be removed from the ethylene stream prior to the oligomerization step. In one embodiment, the carbon monoxide is removed via an oxidation reaction over a copper oxide adsorbent to produce carbon dioxide with the continuous renewal of the spent elemental copper adsorbent back to copper oxide through oxidation by the addition of oxygen. The addition of oxygen also allows conversion of carbon monoxide and hydrogen over available elemental copper sites to carbon dioxide and water. In determining the appropriate amount of oxygen to be added, it is necessary to take into consideration that the hydrogen that is present will also react with the adsorbent. In order to provide a longer adsorbent life, oxygen is co-fed at appropriate levels for continuous regeneration.

The process results in the ability to run the ethanol to jet fuel process for longer periods of time without requiring reload of oligomerization catalysts due to carbon monoxide poisoning of the catalyst. This improves the economics of the process. In addition, the ability to continuously regenerate the CuO catalyst allows the catalyst to be used for longer periods of time without reloading.

In another embodiment, there is presented a three-bed system using a copper containing material in which in one bed CO and H₂ are converted to CO₂ and H₂O in the absence of O₂ to reduce the copper oxide to copper, in another bed any remaining CO and H₂ is converted to CO₂ and H₂O in the presence of O₂ such that the CuO remains oxidized and in a third bed excess O₂ is removed by reaction with reduced Cu. The first and third beds are periodically switched to maintain the O₂ removal capability of the third bed.

### BRIEF DESCRIPTION OF THE DRAWINGS

FIG. 1 shows a simplified flow scheme of the system to produce a clean light olefin stream for oligomerization.

FIG. 2 shows step 1 of a flow scheme with a three-bed system for removing carbon monoxide from an ethylene stream with first bed in lead position regenerating, second bed removing carbon monoxide and hydrogen and third bed in lag position scavenging excess oxygen.

FIG. 3 shows step 2 of a flow scheme with a three-bed system for removing carbon monoxide from an ethylene stream with first bed offline ready to scavenge oxygen, second bed removing carbon monoxide and hydrogen and third bed in lag position scavenging oxygen.

FIG. 4 shows step 3 of a flow scheme with a three-bed system for removing carbon monoxide from an ethylene stream with first bed in lag position scavenging excess oxygen, second bed removing carbon monoxide and hydrogen and third bed offline and ready for regeneration.

FIG. 5 shows step 4 of a flow scheme with a three-bed system for removing carbon monoxide from an ethylene stream with first bed in lag position scavenging excess oxygen, second bed removing carbon monoxide and hydrogen and third bed in lead position regenerating.

FIG. 6 shows step 5 of a flow scheme with a three-bed system for removing carbon monoxide from an ethylene stream with first bed scavenging excess oxygen, second bed removing carbon monoxide and hydrogen and third bed in lead position regenerated.

FIG. 7 shows step 6 of a flow scheme with a three-bed system for removing carbon monoxide from an ethylene stream with first bed offline ready to be regenerated, second bed removing carbon monoxide and hydrogen and third bed in lag position scavenging oxygen.

FIG. 8 shows step 7 of a flow scheme with a three-bed system for removing carbon monoxide from an ethylene stream with first bed being regenerated, second bed removing carbon monoxide and hydrogen and third bed in lag position scavenging oxygen.

### DETAILED DESCRIPTION

The process of producing jet fuel from ethanol involves the key steps of ethanal dehydration to produce ethylene and the ethylene oligomerization to produce the longer chain molecules that are useful for jet fuel range products. It has been found that during the ethanol dehydration process, some carbon monoxide is produced. While constituents such as carbon dioxide and hydrogen, do not inhibit the oligomerization process, carbon monoxide deactivates the oligomerization catalyst. The carbon monoxide level in the ethylene dehydration product is about 200 ppm at the start of the ethanol dehydration catalyst life, can be as high as 700 ppm at the end of the dehydration catalyst life and may increase up to 1500 ppm if methanol is present in the alcohol feed stream to the ethanol dehydration reactor. Methanol is a common denaturant added to ethanol. To increase the cycle time of the oligomerization process, CO must be removed prior to sending to sending the ethylene stream to oligomerization steps to protect the oligomerization catalyst. In a proposed scheme, a vessel containing carbon monoxide removing materials is installed upstream of drier vessels. The carbon monoxide removing materials that may be used include copper containing materials such as copper oxide. The carbon monoxide removing materials can be any metal oxide on porous supports such as activated carbon, zeolites, mesoporous silica, alumina, and metal-organic frameworks which selectively adsorbs CO from the product gas stream. Preliminary experiments discussed herein were conducted with a copper oxide adsorbent. A copper oxide material with oxygen injection was tested to remove CO from the product gas. However, the product stream has about 300 ppm hydrogen, which competes with CO to reduce CuO to Cu, making oxidized copper (CuO) unavailable for CO removal. Hence, oxygen can be injected to the stream and utilize the pre-reduced adsorbent. Since copper reduces to its metallic state, it acts as a catalyst for hydrogen to water conversion. The effluent stream from the dehydration unit contains about 98 wt % ethylene and the remainder comprises of carbon monoxide, carbon dioxide, hydrogen, olefins and oxygenates. Carbon monoxide is a poison to the catalyst in the oligomerization step at the expected ppm levels and must be removed from the ethylene stream before the oligomerization step of the process. The CO concentration is about 200 ppm at the start of a run and can be as high as 700 ppm at the end of the run and may increase up to 1500 ppm if methanol is present as a common denaturant added to ethanol. However, other byproducts such as carbon dioxide and hydrogen do not interfere with the oligomerization catalyst. The reactions are as follows, CO+CuO→Cu+CO2, CuO+H2→Cu+H2O (Activation/Reduction: Pretreatment/Regeneration), CO+CuO→Cu+CO2, H2+CuO→Cu+H2O, ½ O2+Cu→CuO, (Excess O2 is used for CO and H2 removal, and ½ O2+Cu→CuO in an excess O2 scavenging bed.

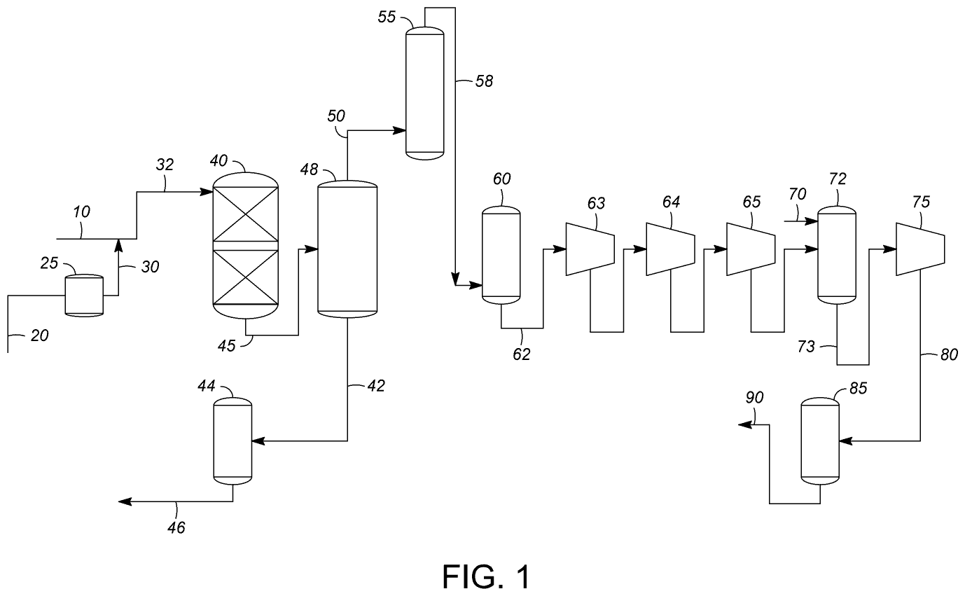

Both a simplified flow scheme such as in FIG. 1 is shown as well as a three bed system that takes into account the need for a bed to be regenerated, a bed to have reactions with carbon monoxide and a bed to recover excess oxygen. Current uses of the catalyst or adsorbent are limited to it either being used in its reduced state as an oxygen scavenger, or in its oxidized state to convert CO to CO2. In both cases, the adsorbent is either replaced or regenerated once it is spent (“spent” in this case means the adsorbent is oxidized in oxygen scavenger service or is reduced in CO oxidation service). Regeneration requires the vessel of adsorbent be removed from service and a regeneration procedure performed with an external stream, which adds operational and capital cost to a design. As described earlier, high levels of CO and H2 in the feed compared to other applications would result in either unreasonably large adsorbent beds, expensive equipment (and electricity and nitrogen use) to perform frequent regenerations, or some combination of these two. This is undesirable from the standpoint of process economics and environmental footprint. This disclosure utilizes a novel process flow scheme combining both functions of the adsorbent to allow for sustained removal of CO from the feed without requiring external streams or extra equipment for regeneration, while meeting the required product specifications (specifically, removal of CO without O2 being left in the product).

By arranging a bed of reduced adsorbent downstream of a bed of oxidized adsorbent, and injecting O2 (as air or pure O2) into the feed to the bed of oxidized adsorbent, complete conversion of CO and H2 can occur via reactions 1 and 2 without depleting the CuO, because the excess O2 will keep the adsorbent oxidized via reaction 3. Then, the excess oxygen leaving the first bed reacts with the reduced adsorbent via reaction 3 in the second bed, and the product is free of CO and O2. The injection of O2 can be controlled by use of analyzers on the feed stream to measure CO, H2, and O2. The actual ratio of O2 to CO+H2 is maintained at the desired target ratio (which may vary from approximately 1.05 to 1.95 parts O2 to 2 parts CO+H2) by controlling the setpoint on the flow controller on the O2 injection line. After some time, the second bed will become spent by oxidation of the Cu. To regenerate (reduce) this bed of adsorbent can be put into service upstream of the first bed.

The CO and H2 in the feed stream will react with the CuO in this adsorbent to reduce it to Cu. Meanwhile, this will reduce the amount of CO and H2 at the inlet to the second bed, so the control scheme described above will reduce the O2 flow rate. A third bed of reduced adsorbent can be installed downstream of the bed of oxidized adsorbent so that oxygen scavenging of the effluent is maintained. The attached drawings show how this three-bed flow scheme can be designed to allow for continuous operation. The first and third bed positions are switched periodically between oxygen scavenging (lag) and regeneration (lead) operation, and the oxidized bed of adsorbent remains in operation between the other two beds. The valves shown could be actuated electronically or pneumatically, with all of the control functions described being performed by a programmable logic controller. The size of the two oxygen scavenging beds would be the same. Because the O2 flow rate is always set to less than 2× the theoretical amount (as described above) the lead bed being regenerated will always be reduced by the feed more quickly than the lag bed is spent. This will prevent oxygen breakthrough from the last bed. A heater or heat exchanger may be required on the feed stream to heat the gas as shown on the figure to the required temperature for the reactions to proceed. Additional heaters, coolers, or heat exchangers not shown on the figure may be required between the adsorbent beds if the optimal temperature for regeneration (reduction), CO+H2 oxidation, and O2 scavenging differ from each other.

FIG. 1 shows a simplified flow scheme showing an ethanol stream being dehydrated to an ethylene stream and then treated to remove carbon monoxide and dried with the clean ethylene routed to an oligomerization and dimerization unit. Ethanol stream 10 is shown being sent to dehydration reactor 40 together with water stream 20 being heated by preheater 25 with the resultant steam 30 being combined with ethanol stream 10. A reactor effluent stream 45 is shown exiting dehydration reactor 40 going to a separator column 48 to be separated into gas stream 50 and liquid stream 42. Liquid stream 42 is sent to water stripper 44 to produce water stream 46 that may be reused. Gas stream 50 that comprises about 99.5% ethylene with CO, CO2 and H2 impurities is sent to DEE adsorber 55 with treated vapor 58 then sent to wash water tower 60 to line 62 and a series of compressors 63. 64 and 65. A supply of oxygen 70 is added to adsorbent bed 72 containing an adsorbent such as cupric oxide to remove CO from the ethylene stream. The purified ethylene 73 is sent to compressor 75 through line 80 to drier 85 containing an adsorbent such as a 3A/13X zeolite mixture with the clean ethylene stream 90 then sent to an oligomerization and dimerization unit (not shown).

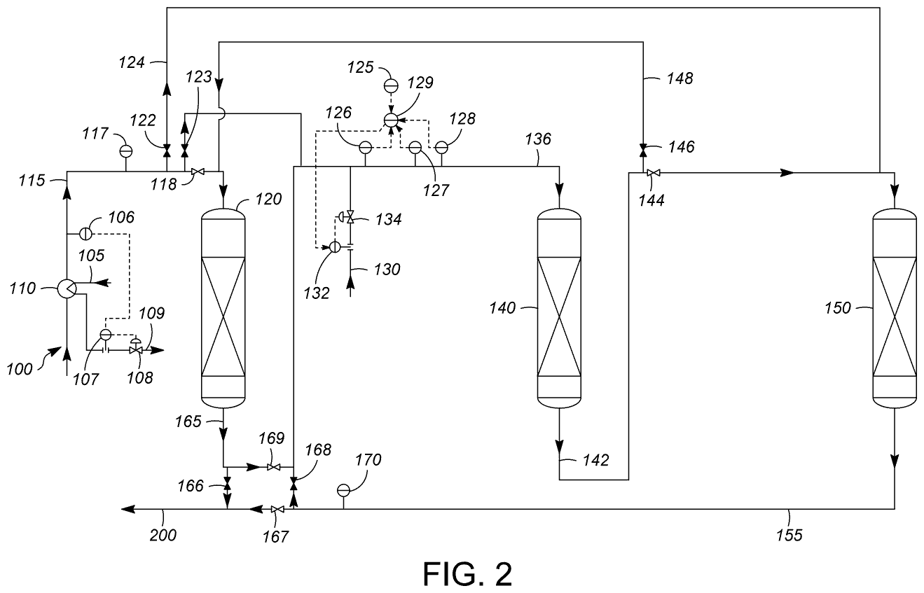

FIGS. 2-8 illustrate a 7-step cycle that is employed in an embodiment of the disclosure in which there is a three adsorbent bed system. In each of the descriptions of the steps, the beds will be referred to by element number or by reference to bed A (120), bed B (140) and bed C (150) from left to right. In the first step shown in FIG. 2, Bed A is reduced and the CO concentration at inlet and outlet is equal meaning that regeneration is complete and the bed can be moved from lead to lag position. Bed B is oxidizing CO and H2 with excess O2 addition. Bed C is partially oxidized and scavenging excess oxygen. No reactions are occurring in bed A. In bed B the reactions occurring include CO+Cu->Cu+CO2, H2+CuO->Cu+H2) and ½ O2+Cu->CuO. In bed C, ½ O2+Cu->CuO. In a first step, a feed 100 is shown being heated by a source of heat such as steam through valve 110. The steam flow is controlled by readings from temperature indication control 106 and flow indication control 107. A portion of steam may be vented through valve 108 and exits at 109. The heated feed 115 is sent to bed 120 through open valve 118. A sensor 117 measures the level of carbon monoxide. Valves 122 and 123 are closed. Shown is line 124 that is in place to send feed to bed 150 in a later step. The stream exits bed 120 through line 165 through open valve 169 to continue to bed 140. A CO treating air control system is shown with sensors 126 (H2), 127 (CO) and 128 (02) to sensor 129 and the stoichiometric ratio of O2 maintained by control 125. Air or oxygen 130 enters with a flow indication control 132 controlling valve 134, shown in open position. The feed goes to bed 140 and the treated feed is sent through line 142 through valve 144 in open position back to bed 150 and then through valve 167 in open position to product 200 exiting to be sent to the oligomerization and dimerization unit (not shown). A sensor 170 measures the amount of CO. Also shown are closed valves 166 and 168. A line 155 is provided so that the effluent of bed 150 may pass through valve 167 to product 200.

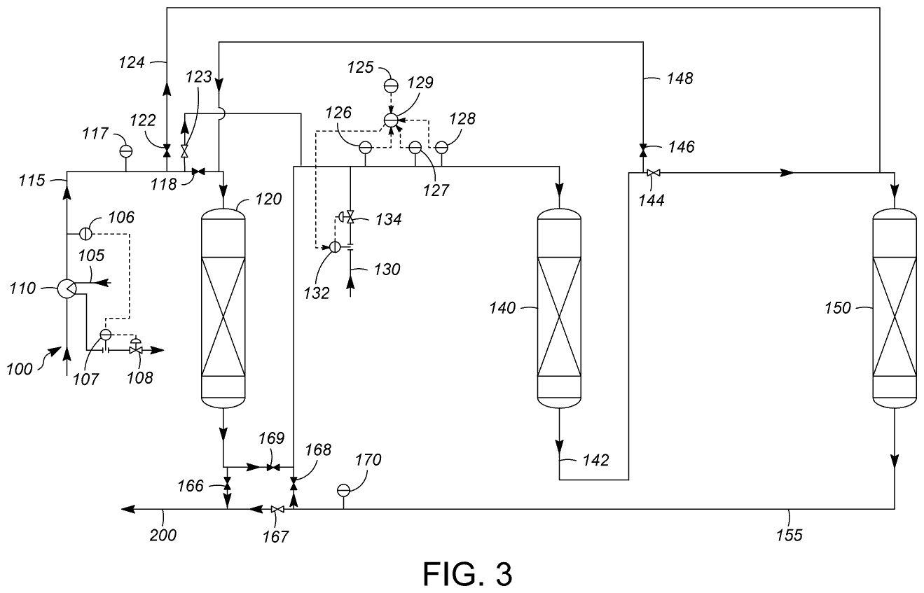

In FIG. 3 is shown step 2 of 8 in which an open bypass valve 123 directs the feed to bed 140. Bed 120 is now isolated with its valves shut. CO and H2 are still being fully oxidized in bed 140 with excess oxygen and bed 150 is still scavenging excess oxygen. Bed 120 is offline with the adsorbent reduced and ready to be put into scavenger service. In bed 140 the reactions occurring include CO+Cu->Cu+CO2, H2+CuO->Cu+H2) and ½ O2+Cu->CuO. In bed C, ½ O2+Cu->CuO. In a third step, a feed 100 is shown being heated by a source of heat such as steam through valve 110. The steam flow is controlled by readings from temperature indication control 106 and flow indication control 107. A portion of steam may be vented through valve 108 and exits at 109. The heated feed 115 bypasses bed 120 with valve 118 closed. A sensor 117 measures the level of carbon monoxide. Valves 123 is open and valve 122 is closed. The feed continues to bed 140. A CO treating air control system is shown with sensors 126 (H2), 127 (CO) and 128 (02) to sensor 129 and the stoichiometric ratio of O2 maintained by control 125. Air or oxygen 130 enters at 130 with a flow indication control 132 controlling valve 134, shown in open position. The feed goes to bed 140 and the treated feed is sent through line 142 through valve 144 in open position to bed 150 and then through valve 167 in open position to product 200 exiting to be sent to the oligomerization and dimerization unit (not shown). A sensor 170 measures the amount of CO. Also shown are closed valves 166, 168 and 169 since bed 120 is offline. Also shown are closed valves 166 and 168. A line 155 is provided so that the effluent of bed 150 may pass through valve 167 to product 200.

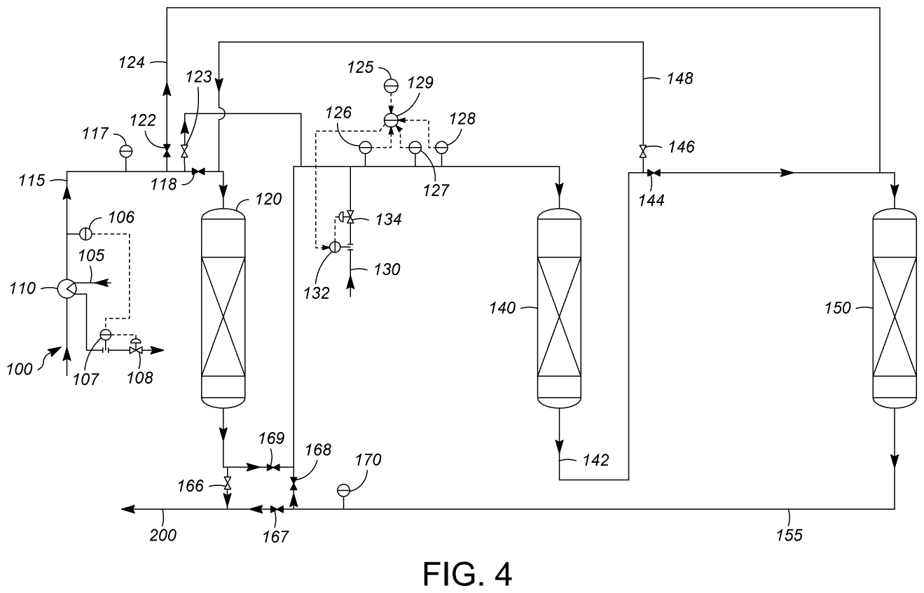

In FIG. 4 is shown step 3 of 7 in which bed 120 is in lag position by opening a valve from bed 140 outlet to the bed 120 inlet. A valve from bed 120 is opened to go to product. Then valves in and out of bed 150 are closed to take that bed offline. Bed 120 is now the oxygen scavenging lag bed, bed 140 is still reacting the CO and H2 with excess oxygen and bed 150 is isolated. More specifically, an open bypass valve 123 directs the feed to bed 140. Bed 120 is now isolated with its valves shut. CO and H2 are still being fully oxidized in bed B with excess oxygen and bed C is still scavenging excess oxygen. Bed 120 is now the oxygen scavenging bed with ½ O2+Cu->CuO. Bed 140 the reactions occurring include CO+Cu->Cu+CO2, H2+CuO->Cu+H2) and ½ O2+Cu->CuO. Bed 150 is offline. In a fourth step, a feed 100 is shown being heated by a source of heat such as steam through valve 110. The steam flow is controlled by readings from temperature indication control 106 and flow indication control 107. A portion of steam may be vented through valve 108 and exits at 109. The heated feed 115 bypasses bed 120 with valve 118 closed. A sensor 117 measures the level of carbon monoxide. Valve 123 is open and valve 122 is closed. The feed to bed 140. A CO treating air control system is shown with sensors 126 (H2), 127 (CO) and 128 (02) to sensor 129 and the stoichiometric ratio of O2 maintained by control 125. Air or oxygen 130 enters at 130 with a flow indication control 132 controlling valve 134, shown in open position. The feed goes to bed 140 and the treated feed is sent through valves 146 to line 148 back to bed 120 and then through valve 166 in open position to product 200 exiting to be sent to the oligomerization and dimerization unit (not shown). A sensor 170 measures the amount of CO. Also shown are closed valves 167 and 168. Valve 144 is closed so that flow is not being sent to bed 150 at this time. Also shown are closed valves 166 and 168. A line 155 is provided so that the effluent of bed 150 may pass through closed valve 167 to product 200 when bed 150 is back in operation.

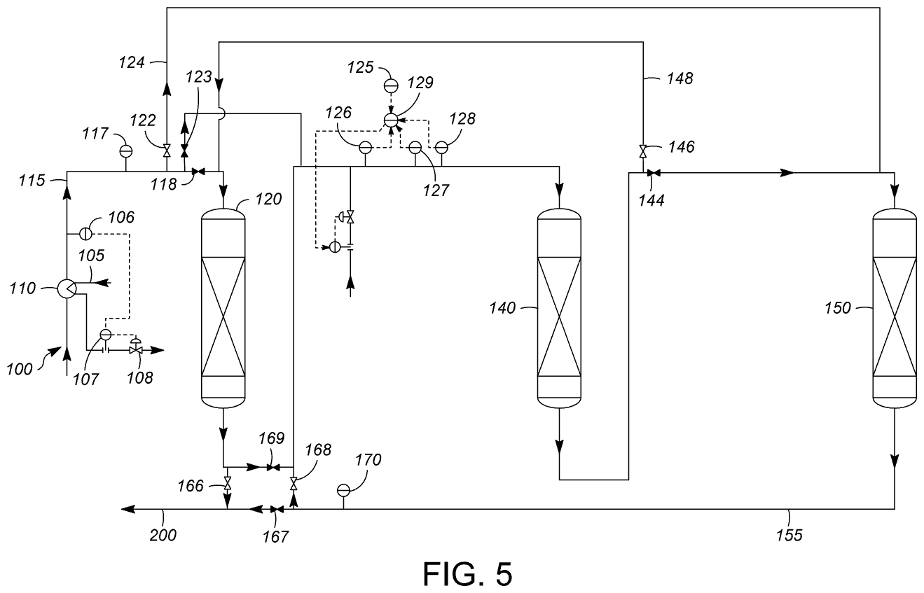

In FIG. 5 is shown step 4 of the 7-step process. Bed 150 is in lead regeneration position by opening the feed valve 146 to bed 150 and opening valve 168 connected to the outlet flow from bed 150. Then the bypass valve 123 is closed. As the CO and H2 start to be consumed by bed 150, the oxygen requirement will go down and the oxygen flow will be decreased. a feed 100 is shown being heated by a source of heat such as steam through valve 110. The steam flow is controlled by readings from temperature indication control 106 and flow indication control 107. A portion of steam may be vented through valve 108 and exits at 109. The heated feed 115 bypasses bed 120 with valve 118 closed. A sensor 117 measures the level of carbon monoxide. Valves 123 is open and valve 122 is closed. The feed continues to bed 140. A CO treating air control system is shown with sensors 126 (H2), 127 (CO) and 128 (02) to sensor 129 and the stoichiometric ratio of O2 maintained by control 125. Air or oxygen 130 enters at 130 with a flow indication control 132 controlling valve 134, shown in open position. The feed goes to bed 140 and the treated feed is sent through line 142 through valve 146 to return to bed 120 and then flow through valve 166 to the product 200 that is sent to the dimerization and oligomerization reactor. Valve 144 is closed so at this point the flow is not being sent to bed 150. in open position to bed 150 and then through valve 167 in open position to product 200 exiting to be sent to the oligomerization and dimerization unit (not shown). A sensor 170 measures the amount of CO. Also shown is open valve 168.

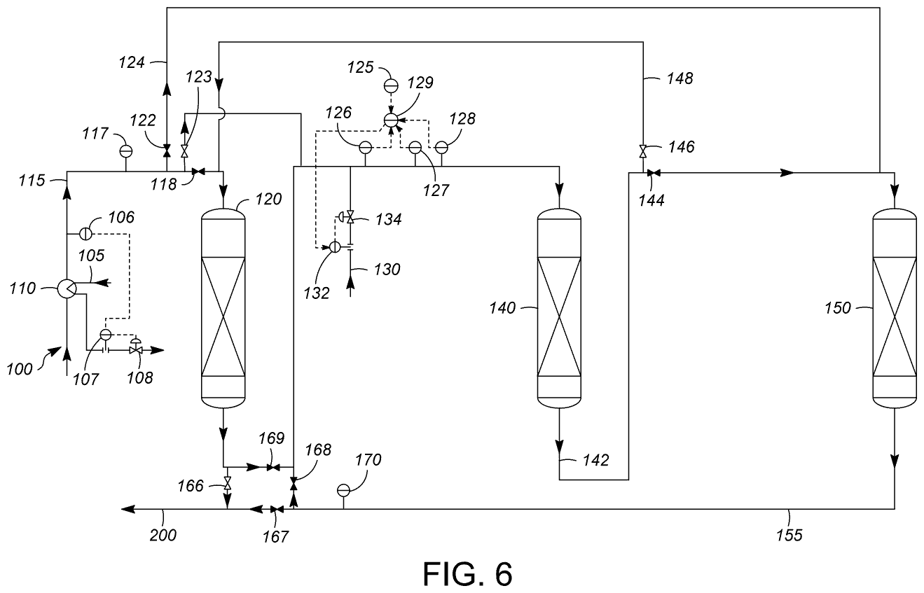

In FIG. 6 is shown step 5 of the 7-step process. Once the CO concentration at the inlet and outlet of bed 150 are equal, the bed is full regenerated cand can be placed back in the lag position. The bypass valves 146 is opened and the valves to bed 150 are closed. A feed 100 is shown being heated by a source of heat such as steam through valve 110. The steam flow is controlled by readings from temperature indication control 106 and flow indication control 107. A portion of steam may be vented through valve 108 and exits at 109. The heated feed 115 bypasses bed 120 with valve 118 closed. A sensor 117 measures the level of carbon monoxide. Valve 123 is open and valve 122 is closed. The feed continues to bed 140. A CO treating air control system is shown with sensors 126 (H2), 127 (CO) and 128 (02) to sensor 129 and the stoichiometric ratio of O2 maintained by control 125. Air or oxygen 130 enters at 130 with a flow indication control 132 controlling valve 134, shown in open position. The feed goes to bed 140 and the treated feed is sent through line 142 through open valve 146 to return to bed 120 and then flow through open valve 166 to the product 200 that is sent to the dimerization and oligomerization reactor. Valve 144 is closed so at this point the flow is not being sent to bed 150. Valve 168 is now closed. Also shown are closed valves 166, 167 and 168. A line 155 is provided so that the effluent of bed 150 may pass through valve 167 to product 200.

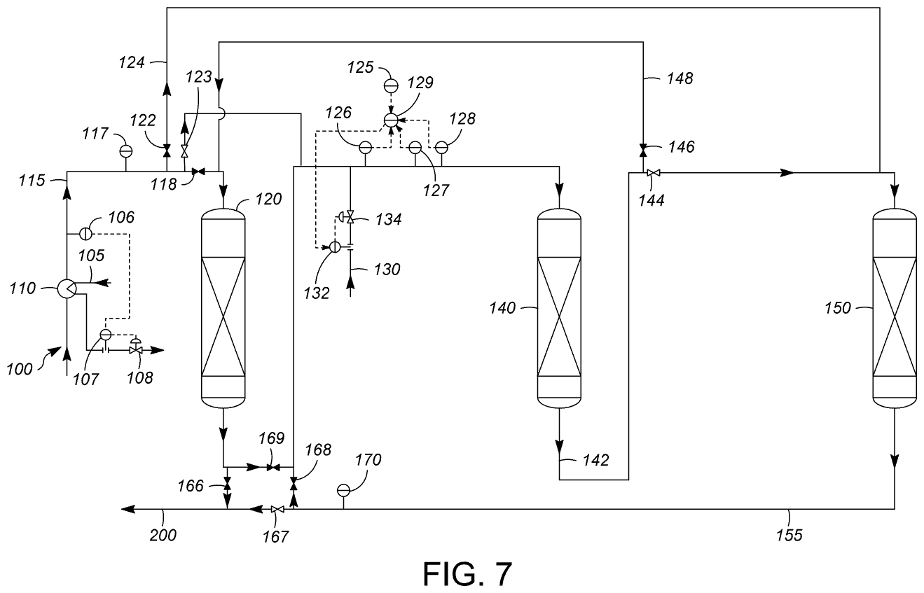

In FIG. 7 is shown step 6 of the 7-step process. Regenerated bed 150 is back in lag position by opening of valve 144 from the outlet of bed 140 and bed 150 has its effluent flow through open valve 167 to product 200. Bed 120 is offline, partially oxidized and ready for regeneration, bed 140 has an excess of O2 to be used in removal of CO and H2 and bed 150 is functioning as a scavenger of excess oxygen. A feed 100 is shown being heated by a source of heat such as steam through valve 110. The steam flow is controlled by readings from temperature indication control 106 and flow indication control 107. A portion of steam may be vented through valve 108 and exits at 109. The heated feed 118 bypasses bed 120 with valve 118 closed so that feed may travel to bed 140. A sensor 117 measures the level of carbon monoxide. Valve 123 is open and valve 122 is closed. The feed continues to bed 140. A CO treating air control system is shown with sensors 126 (H2), 127 (CO) and 128 (02) to sensor 129 and the stoichiometric ratio of O2 maintained by control 125. Air or oxygen 130 enters with a flow indication control 132 controlling valve 134, shown in open position. The feed goes to bed 140 and the treated feed is sent through line 142 through open valve 144 to go to bed 150 and then flow through open valve 167 to the product 200 that is sent to the dimerization and oligomerization reactor. Valves 166 and 168 are closed so at this point the flow is not being sent to any other bed. Also shown are closed valves 166 and 168. A line 155 is provided so that the effluent of bed 150 may pass through valve 167 to product 200.

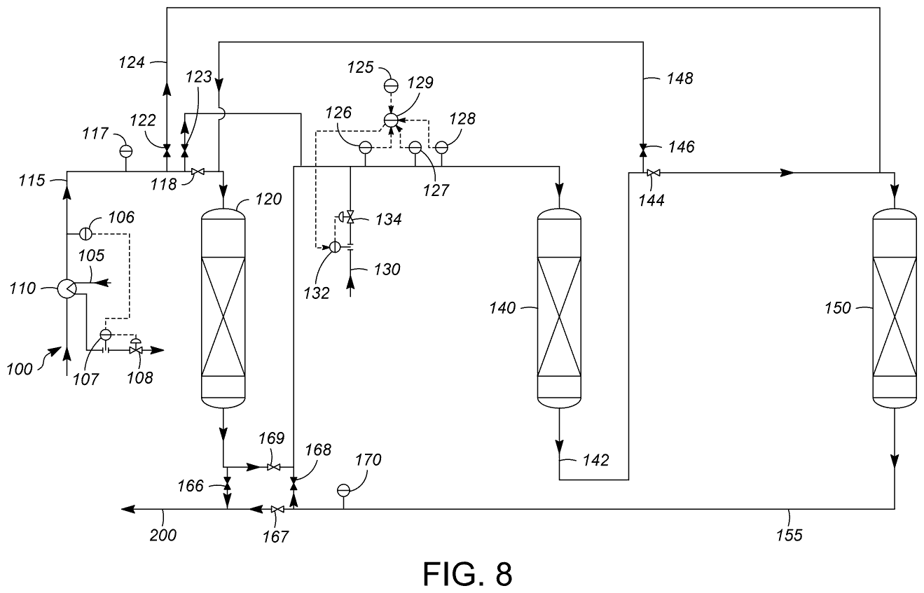

In FIG. 8 is shown step 7 of the 7-step process. Bed 120 is in lead position to begin regeneration by opening the valves to and from the bed and the configuration is the same as step 1 shown in FIG. 1A feed 100 is shown being heated by a source of heat such as steam through valve 110. The steam flow is controlled by readings from temperature indication control 106 and flow indication control 107. A portion of steam may be vented through valve 108 and exits at 109. The heated feed 118 goes to bed 120 with valve 118 open. A sensor 117 measures the level of carbon monoxide. Valve 122 and valve 122 are closed. The feed continues to bed 140. A CO treating air control system is shown with sensors 126 (H2), 127 (CO) and 128 (02) to sensor 129 and the stoichiometric ratio of O2 maintained by control 125. Air or oxygen 130 enters with a flow indication control 132 controlling valve 134, shown in open position. The feed goes to bed 140 and the treated feed is sent through line 142 through open valve 144 to go to bed 150 and then stream 155 flow through open valve 167 to the product 200 that is sent to the dimerization and oligomerization reactor. Valves 166 and 168 are closed so at this point the flow is not being sent to any other bed.

### Examples 1-6

To demonstrate the removal of CO from an ethylene stream in the presence of H2, a series of experiments were conducted using a copper-based adsorbent. The conditions of these experiments are detailed in Table 1. A series of lab-scale experiments were conducted to test the effectiveness of a copper-based adsorbent in removing CO from a C2=stream in the presence of H2.

Example 1 showed that when the feed composition contained only CO/CO2 and N2, the copper adsorbent completely removed CO with no breakthrough observed for 30 hours.

However, in Example 2, when H2 was introduced along with CO, breakthrough occurred early as the adsorbent reacted with both CO and H2 in competing reactions. In experiment 1092, oxygen was introduced at 2.3 times the stoichiometric ratio relative to CO+H2 contaminants, resulting in no breakthrough for 30 hours.

Further investigation of the oxygen concentration in excess or sub-stoichiometric levels relative to CO+H2 contaminants resulted in no observable CO breakthrough for 20 and 18 hours respectively (Examples 3 and 4).

Finally, in Example 5, nitrogen was substituted with ethylene to prove the concept would work in the desired feed environment at sub-stoichiometric O2 concentrations relative to CO+H2 contaminants. No CO breakthrough was observed for 25 hours.

TABLE 1
Ethylene stream CO removal

| | Example | 1 | 2 | 3 | 4 | 5 | 6 |
|---|---|---|---|---|---|---|---|
| | Variables | Temp. | H2 | O2 addition 2.3x | O2 addition 1.5x | O2 addition 0.75x | C2═ |
| | | | addition | stoichiometric | stoichiometric | stoichiometric | addition |
| | Adsorbent | 14.5 | 14.5 | 14.5 | 14.5 | 14.5 | 14.5 |
| | loading, cc | | | | | | |
| Feed (vol %) | CO | 0.07 | 0.07 | 0.07 | 0.07 | 0.07 | 0.07 |
| | CO2 | 0.13 | 0.13 | 0.13 | 0.13 | 0.13 | 0.13 |
| | O2 | 0.00 | 0.00 | 1.25 | 0.80 | 0.40 | 0.40 |
| | H2 | 0.00 | 1.00 | 1.00 | 1.00 | 1.00 | 1.00 |
| | N2 | 99.8 | 98.8 | 97.55 | 98.00 | 98.4 | 6.10 |
| | C2═ | 0.00 | 0.00 | 0.00 | 0.00 | 0.00 | 92.30 |
| | Total | 100.00 | 100.00 | 100.00 | 100.00 | 100.00 | 100.00 |
| Process | Temperature, C. | 110-150 | 150 | 150 | 150 | 150 | 150 |
| Conditions | Pressure, psig | 485 | 485 | 485 | 485 | 485 | 485 |
| | sccm | 87 | 87 | 87 | 87 | 87 | 87 |
| | GHSV (at | 16 | 16 | 16 | 16 | 16 | 16 |
| | condition), hr−1 | | | | | | |
| | CO breakthrough | N (30 hrs) | Y (7 hrs) | N (30 hrs) | N (20 hrs) | N (18 hrs) | N (25 hrs) |

Table 2 represents feed and product effluent samples that were taken for offline composition analysis. The offline analysis shows that the hydrogenation of ethylene to ethane has taken place, but to a minimal extent and is consistent with the online data. The experiment was carried out for approximately 25 hours, with ethylene present and 02 0.75 times the stoichiometric ratio relative to CO+H2 contaminants present in the feed. It has been observed that there is an initial induction period in which CO2 is completely adsorbed onto the copper-containing adsorbent. Once CO2 breakthrough is reached, it can be seen at 22 hours on stream that the increase in CO2 concentration corresponds to the sum of the feed CO2 concentration plus the expected increase in CO2 if all CO is converted at 22 hours on stream.

TABLE 2
Hours on stream vs Concentration via Offline Analysis

| Hours on Stream | CO ppm | CO₂ ppm | C₂H₆ ppm |
|---|---|---|---|
| FEED | 670 | 1200 | 200 |
| 4 | 0 | 10 | 600 |
| 8 | 0 | 800 | 500 |
| 22 | 0 | 1800 | 600 |

### Specific Embodiments

While the following is described in conjunction with specific embodiments, it will be understood that this description is intended to illustrate and not limit the scope of the preceding description and the appended claims.

A first embodiment of the invention is a process for purifying a stream containing light olefins, hydrogen, and carbon monoxide by sending said stream and a second stream comprising of oxygen to one or more vessels to contact a copper-containing material to remove carbon monoxide and hydrogen and produce a stream comprising carbon dioxide and oxygen-free light olefins. An embodiment of the invention is one, any or all of prior embodiments in this paragraph up through the first embodiment in this paragraph wherein the light olefin is ethylene. An embodiment of the invention is one, any or all of prior embodiments in this paragraph up through the first embodiment in this paragraph wherein a volume of said oxygen is at less than 100% stoichiometric level compared to said carbon monoxide and said hydrogen. An embodiment of the invention is one, any or all of prior embodiments in this paragraph up through the first embodiment in this paragraph wherein the bed is at a temperature of about 60° C. to about 175° C. An embodiment of the invention is one, any or all of prior embodiments in this paragraph up through the first embodiment in this paragraph wherein the bed is at a temperature of about 110° C. to about 150° C. An embodiment of the invention is one, any or all of prior embodiments in this paragraph up through the first embodiment in this paragraph wherein at least 95 vol % of said oxygen is converted to water or carbon dioxide within the vessel. An embodiment of the invention is one, any or all of prior embodiments in this paragraph up through the first embodiment in this paragraph wherein the stream is from an alcohol dehydration reactor. An embodiment of the invention is one, any or all of prior embodiments in this paragraph up through the first embodiment in this paragraph wherein the feed stream comprises at least 50 ppm CO. An embodiment of the invention is one, any or all of prior embodiments in this paragraph up through the first embodiment in this paragraph wherein the feed stream comprises at least 200-2000 ppm CO. An embodiment of the invention is one, any or all of prior embodiments in this paragraph up through the first embodiment in this paragraph wherein the product stream comprises less than 10 ppm CO. An embodiment of the invention is one, any or all of prior embodiments in this paragraph up through the first embodiment in this paragraph wherein the vessel is operated at a pressure of about 200-800 psig and a GSHV of about 200-10,000 hr-1. An embodiment of the invention is one, any or all of prior embodiments in this paragraph up through the first embodiment in this paragraph wherein the carbon monoxide and the hydrogen react with the copper-containing material or oxygen to produce carbon dioxide and water.

A second embodiment of the invention is a process for removing carbon monoxide from a stream comprising light olefin, hydrogen, and carbon monoxide comprising sending said stream to a first vessel to contact a copper-containing material to produce a product stream comprising light olefin, carbon dioxide, and water wherein said copper-containing material is first reduced to copper with said hydrogen and carbon monoxide and oxidized by contact with said oxygen. An embodiment of the invention is one, any or all of prior embodiments in this paragraph up through the first embodiment in this paragraph wherein a flow controller controls a volume of oxygen in said second stream so that a ratio of oxygen to carbon monoxide and hydrogen is maintained at a ratio from about 1.05 to 1.95 parts oxygen to 2 parts carbon monoxide plus hydrogen within said vessel. An embodiment of the invention is one, any or all of prior embodiments in this paragraph up through the first embodiment in this paragraph wherein when the copper-containing material is in a reduced state in said first vessel, the stream is sent to a second vessel wherein the copper-containing material is in an oxidized state and oxygen is sent to the first vessel until the copper-containing material has reached an oxidized state. An embodiment of the invention is one, any or all of prior embodiments in this paragraph up through the first embodiment in this paragraph wherein heat exchangers, coolers and heaters maintain a predetermined temperature for said vessel through oxidation and reduction reactions.

A third embodiment of the invention is a system comprising a first bed at conditions to regenerate a copper-containing material from a reduced state to an oxidized state wherein said first bed is configured to receive a stream comprising light olefins, hydrogen, and carbon monoxide; a second bed containing a copper-containing material at conditions to receive a first stream comprising carbon monoxide, light olefins and hydrogen and a second stream comprising oxygen at conditions to convert carbon monoxide to carbon dioxide and hydrogen to water; and a third bed downstream of said second bed containing reduced copper-containing material to be oxidized by unconverted oxygen from said second bed. An embodiment of the invention is one, any or all of prior embodiments in this paragraph up through the first embodiment in this paragraph further comprising a control system on said second bed to control the second stream of oxygen at volumes to convert at least 95% of said carbon monoxide to carbon dioxide while minimizing a volume of oxygen exiting the second bed.

1. A process for purifying a stream containing light olefins, hydrogen, and carbon monoxide by sending said stream and a second stream comprising of oxygen to one or more vessels to contact a copper-containing material to remove carbon monoxide and hydrogen and produce a stream comprising carbon dioxide and oxygen-free light olefins.

2. The process of claim 1 wherein the light olefin is ethylene.

3. The process of claim 1 wherein a volume of said oxygen is at less than 100% stoichiometric level compared to said carbon monoxide and said hydrogen.

4. The process of claim 1 wherein said bed is at a temperature of about 60° C. to about 175° C.

5. The process of claim 1 wherein said bed is at a temperature of about 110° C. to about 150° C.

6. The process of claim 1 wherein at least 95 vol % of said oxygen is converted to water or carbon dioxide within said vessel.

7. The process of claim 1 wherein said stream is from an alcohol dehydration reactor.

8. The process of claim 1 wherein said feed stream comprises at least 50 ppm CO.

9. The process of claim 1 wherein said feed stream comprises at least 200-2000 ppm CO.

10. The process of claim 1 wherein said product stream comprises less than 10 ppm CO.

11. The process of claim 1 wherein said vessel is operated at a pressure of about 200-800 psig and a GSHV of about 200-10,000 hr-1.

12. The process of claim 1 wherein said carbon monoxide and said hydrogen react with said copper-containing material or oxygen to produce carbon dioxide and water.

13. A process for removing carbon monoxide from a stream comprising light olefin, hydrogen, and carbon monoxide comprising sending said stream to a first vessel to contact a copper-containing material to produce a product stream comprising light olefin, carbon dioxide, and water wherein said copper-containing material is first reduced to copper with said hydrogen and carbon monoxide and oxidized by contact with said oxygen.

14. The process of claim 13 wherein a flow controller controls a volume of oxygen in said second stream so that a ratio of oxygen to carbon monoxide and hydrogen is maintained at a ratio from about 1.05 to 1.95 parts oxygen to 2 parts carbon monoxide plus hydrogen within said vessel.

15. The process of claim 13 wherein when said copper-containing material is in a reduced state in said first vessel, said stream is sent to a second vessel wherein said copper-containing material is in an oxidized state and oxygen is sent to said first vessel until said copper-containing material has reached an oxidized state.

16. The process of claim 12 wherein heat exchangers, coolers and heaters maintain a predetermined temperature for said vessel through oxidation and reduction reactions.

17. A system comprising
a. a first bed at conditions to regenerate a copper-containing material from a reduced state to an oxidized state wherein said first bed is configured to receive a stream comprising light olefins, hydrogen, and carbon monoxide;
b. a second bed containing a copper-containing material at conditions to receive a first stream comprising carbon monoxide, light olefins and hydrogen and a second stream comprising oxygen at conditions to convert carbon monoxide to carbon dioxide and hydrogen to water; and
c. a third bed downstream of said second bed containing reduced copper-containing material to be oxidized by unconverted oxygen from said second bed.

18. The system of claim 16 further comprising a control system on said second bed to control said second stream of oxygen at volumes to convert at least 95% of said carbon monoxide to carbon dioxide while minimizing a volume of oxygen exiting said second bed.
